(19) United States
(12) Patent Application Publication
(10) Pub. No.: US 2025/0269015 A1
(43) Pub. Date: Aug. 28, 2025

(54) POLYMERIC ADJUVANT

(71) Applicant: VIROVAX LLC, Lawrence, KS (US)

(72) Inventor: Sunil Abraham David, Lawrence, KS (US)

(21) Appl. No.: 19/062,936

(22) Filed: Feb. 25, 2025

Related U.S. Application Data
(60) Provisional application No. 63/559,075, filed on Feb. 28, 2024.

(51) Int. Cl. A61K 39/39; A61K 39/00; A61P 37/04; C08F 8/32

(52) U.S. Cl. CPC A61K 39/39; A61P 37/04; C08F 8/32; A61K 2039/55511

(57) ABSTRACT

A polymeric adjuvant includes at least one 1-(3-(aminomethyl)benzyl)-2-butyl-1H-imidazo[4,5-c]quinolin-4-amine covalently conjugated to one least one carboxylic acid group of poly (acrylic acid) to form an amide bond, and its sodium and potassium salts thereof. The degree of conjugation of 1-(3-(aminomethyl)benzyl)-2-butyl-1H-imidazo[4,5-c]quinolin-4-amine to poly(acrylic acid) is from about 5% to about 50%. A composition can include the adjuvant and an immunogen in a pharmaceutical carrier (e.g., a buffer).

### CROSS-REFERENCE TO RELATED APPLICATIONS

This patent application claims priority to U.S. Provisional Application No. 63/559,075 filed Feb. 28, 2024, which provisional is incorporated herein by specific reference in its entirety.

### BACKGROUND

### Field

The present disclosure relates to substituted polymeric materials for use as adjuvants for immune responses. More particularly, the substituted polymeric materials include a poly (acrylic acid) substituted with an imidazoquinoline having a meta-aminomethylbenzyl group (1-(3-(aminomethyl)benzyl)-2-butyl-1H-imidazo[4,5-c]quinolin-4-amine) as a covalently-linked appendage.

### Description of Related Art

In order to achieve a high level of efficacy and safety, many newer vaccines that have immunological agents linked to lower immunogenicity rely on potent immunostimulants (e.g., adjuvants). An immunogen is any substance that generates B-cell (humoral/antibody) and/or T-cell (cellular) adaptive immune responses upon exposure to a host organism. Immunogens that generate antibodies are called antigens (“antibody- generating”). The Food and Drug Administration (FDA) considers an adjuvant to be a substance added to vaccines to enhance the immune response in vaccinated individuals. Adjuvants also serve to reduce the amount of antigen needed for the induction of a robust immune response (e.g., ‘dose-sparing effect’) or reduce the number of immunizations needed for protective immunity. The ability of adjuvants to broaden antibody responses could be crucial for the success of vaccines against many pathogens that display substantial antigenic drift and/or strain variations including influenza viruses, human immunodeficiency virus (HIV), human papilloma virus (HPV), and the malaria parasite (e.g., plasmodium parasites). Adjuvants also help improve the efficacy of vaccines in newborns, the elderly or immunocompromised persons.

### SUMMARY

In some embodiments, a polymeric adjuvant includes at least one 1-(3-(aminomethyl)benzyl)-2-butyl-1H-imidazo[4,5-c]quinolin-4-amine covalently conjugated to one least one carboxylic acid group of poly (acrylic acid) to form an amide bond, and its sodium and potassium salts thereof.

In some embodiments, the degree of conjugation of 1-(3-(aminomethyl)benzyl)-2-butyl-1H-imidazo[4,5-c]quinolin-4-amine to poly (acrylic acid) is from about 5% to about 50% of the available monomers having carboxylic acid that can form an amide bond. In some embodiments, the composition can include the adjuvant and an immunogen

(e.g., having an antigen and immunogenicity) in a pharmaceutical carrier (e.g., a buffer). Such a composition can be administered either intramuscularly, subcutaneously, or intranasally to a subject in order to provide antigen-specific immunity.

In some embodiments, the composition can include the adjuvant and an immunogen which has been adsorbed on “alum” (e.g., aluminum hydroxide and/or aluminum phosphate) and a pharmaceutical carrier (e.g., a buffer). Such a composition can be administered intramuscularly.

In some embodiments, the composition can be devoid of alum particles. Such a composition can be administered intranasally, intramuscularly, or subcutaneously.

As used herein, “CQUIM-MA” is defined as at least one 1-(3-(aminomethyl)benzyl)-2-butyl-1H-imidazo[4,5-c]quinolin-4-amine conjugated via the meta-aminomethylbenzyl substituent at position N1 of the imidazoquinoline, to carboxylic acid groups of poly (acrylic acid) to form amid bonds. The CQUIM-MA is a polymeric adjuvant that is fully water-soluble.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 includes a graph illustrating data for a rapid-throughput, high-content, Microfocus Reduction Neutralization Test (MFRNT) that shows the intranasal vaccine construct with the polymeric adjuvant CQUIM-MA elicits very high systemic neutralizing antibodies against live Delta SARS-COV-2.

FIG. 2 includes a graph illustrating data for the MFRNT that shows CQUIM-MA has excellent adjuvant activity with intramuscular vaccine administration, and elicits very high neutralizing antibodies even in the absence of aluminum hydroxide.

FIG. 3 includes a graph illustrating data for the MFRNT that shows CQUIM-MA has excellent adjuvant activity with intramuscular vaccine administration, and has superior adjuvant activity over Alhydroxiquim-II.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology includes the polymeric adjuvant described herein. The polymeric adjuvant can be used to increase adjuvant activity for an immunogen, which increases an immunological response to the immunogen. Accordingly, the polymeric adjuvant can be used to increase an immunological response to an immunogen from pathogens or allergens. In some aspects, the polymeric adjuvant described herein can be used in therapeutic methods for treating, preventing, and/or slowing progression of an infection or allergic reaction. The polymeric adjuvant can be included in compositions with immunogens, or may be included in an adjuvant composition without an immunogen. The polymeric adjuvant can be administered simultaneously with the immunogen, or administered separately. Therefore, the polymeric adjuvant is administered so as to increase the immunological response to the immunogen.

In some embodiments, the polymetric adjuvant includes at least one 1-(3-(aminomethyl)benzyl)-2-butyl-1H-imidazo[4,5-c]quinolin-4-amine conjugated through the meta-aminomethylbenzyl group to one or more carboxylic acid groups of poly (acrylic acid).

In some embodiments, the polymeric adjuvant has the structure of Formula 1:

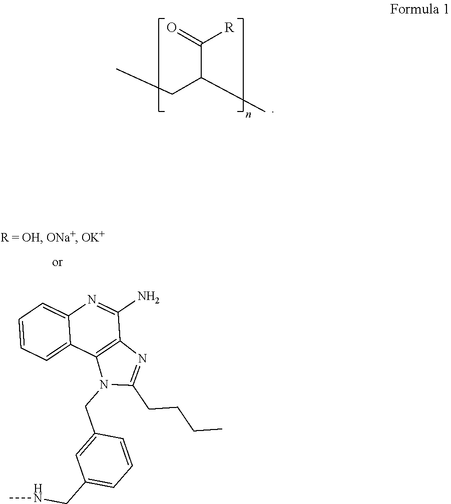

In Formula 1, the polymeric adjuvant includes at least one 1-(3-(aminomethyl)benzyl)-2-butyl-1H-imidazo[4,5-c]quinolin-4-amine conjugated via the meta-aminomethylbenzyl substituent at position N1 of the imidazoquinoline to one or more carboxylic acid groups of poly (acrylic acid). In Formula 1, ‘n’ is an integer. In Formula 1, the unconjugated R groups can be any combination of the hydroxyl or salts at any amounts depending on conditions of the polymeric adjuvant formulation. In some aspects, the salt forms of the polymeric adjuvant include sodium and/or potassium salts.

In some embodiments, a degree of conjugation of 1-(3-(aminomethyl)benzyl)-2-butyl-1H-imidazo[4,5-c]quinolin-4-amine to the carboxyl groups of the poly (acrylic acid) is from about 5% to about 50%.

In Formula 1, the ‘n’ determines the molecular weight of the polymer (e.g., ‘n’ is approximately 5,114 for poly (acrylic acid) of molecular weight 450,000 Da). Accordingly, the ‘n’ can range from about 1,500 to about 11,000.

In some embodiments, a composition containing the polymeric adjuvant can be administered intramuscularly to a subject. Intramuscular administration of compositions, such as vaccines, is well-known in the art.

In some embodiments, a composition containing the polymeric adjuvant can be administered to the nasal cavity of a subject. Intranasal administration of compositions, such as vaccines, is well-known in the art.

In some embodiments, the composition containing the polymeric adjuvant is devoid of “alum” particles. An alum particle is a finely divided solid composed of hydrated aluminum hydroxide, or a phosphate salt, dispersed in water or buffer. In some aspects, an immunological composition having an immunogen being administered with the polymeric adjuvant can be devoid of alum particles. In some aspects, an immunization is performed with the polymeric adjuvant and without alum particles. For example, a composition having the polymeric adjuvant that is devoid of alum particles can be administered intranasally.

In some embodiments, a composition having the polymeric adjuvant that is devoid of alum particles can be lyophilized. Now, the present polymeric adjuvant and immunogen formulated in a pharmaceutical buffer can be lyophilized, thereby prolonging the shelf-life of the immunological composition. The lyophilized composition can be reconstituted in buffer prior to administration.

In some embodiments, the composition having the polymeric adjuvant can contain alum particles. For example, the composition can include an immunogen adsorbed on alum, polymeric adjuvant, and a pharmaceutical carrier (e.g., buffer). In some aspects, immunological compositions containing alum are not lyophilized.

In some embodiments, the present technology includes an adjuvant that can be used for inducing immunity to an immunogen in a subject, whether a viral or a non-viral-derived immunogen. The immunogen can be any type of immunogen that a subject can develop immunity against, such as protein, or peptide. In some aspects, the immunogen can be a purified protein or portion thereof (e.g., antigen portion), or combinations of proteins. In some aspects, the immunogen can be a purified nucleic acid. The immunogen can be isolated from a natural source (e.g., from pollen or venom) or produced in a laboratory setting (e.g., from bacteria or mammalian cells configured to produce the immunogen). In some aspects, the immunogen can be from pollen, venom (e.g., snake, spider, etc.), bacterial, virus, fungus, or the like. The immunogen can be a protein, peptide, or in native form or chemically modified (e.g., modified with formaldehyde, glutaraldehyde, and beta-propiolactone, etc.). The immunogen can include an attenuated pathogen, such as virus, bacteria, or fungus, which includes all or part of the attenuated pathogen. The immunogen can include an inactivated (e.g., killed or dead) pathogen, such as virus, bacteria, or fungus, which includes all or part of the attenuated pathogen. In some aspects, the immunogen can be a protein or polypeptide, or nucleic acid encoding a protein or peptide or at least a portion of a virus, bacteria, or fungus or allergen.

### SYNTHESIS OF ADJUVANT

The coupling of the benzylic amine in 1-(3-(aminomethyl)benzyl)-2-butyl-1H-imidazo[4,5-c]quinolin-4-amine to the carboxylates in poly (acrylic acid) can be accomplished using a variety of coupling agents. A representative example using 1-ethyl-3-(3-dimethylaminopropyl) carbodiimide (EDCI) is as follows.

To a 1 L beaker containing 350 mL of water was added 3.5 g poly (acrylic acid) [MW: 450,000] and stirred for 2 h until complete dissolution was achieved. 350 mg of 1-(3-(aminomethyl)benzyl)-2-butyl-1H-imidazo[4,5-c]quinolin-4-amine (hydrochloride salt) was added and stirred until completely dissolved. 460 mg of EDCI.HCl was then added and stirred until fully dissolved. A pH of 2.33 was observed. Small aliquots of triethylamine were added until a pH of 7.5 was reached. The reaction was allowed to proceed for 8 h.

The product was purified by ion-exchange chromatography using Dowex™ 50WX8 100-200 (H). The pH of the purified material was adjusted to 7.4 with 1N NaOH and lyophilized. The imidazoquinoline content in the polymeric adjuvant compound was measured using spectrophotometry at 332 nm, with linear interpolation of a standard curve constructed using 1-(3-(aminomethyl)benzyl)-2-butyl-1H-imidazo[4,5-c]quinolin-4-amine (hydrochloride salt). The amount of polymeric adjuvant can be expressed as micrograms of imidazoquinoline per milligram of purified lyophilizate (e.g., hydrated, sodium salt form of the adjuvant compound).

### EXAMPLES

### Adjuvant for Intranasal Subunit Vaccines (Rabbit Immunogenicity Trial VV-39)

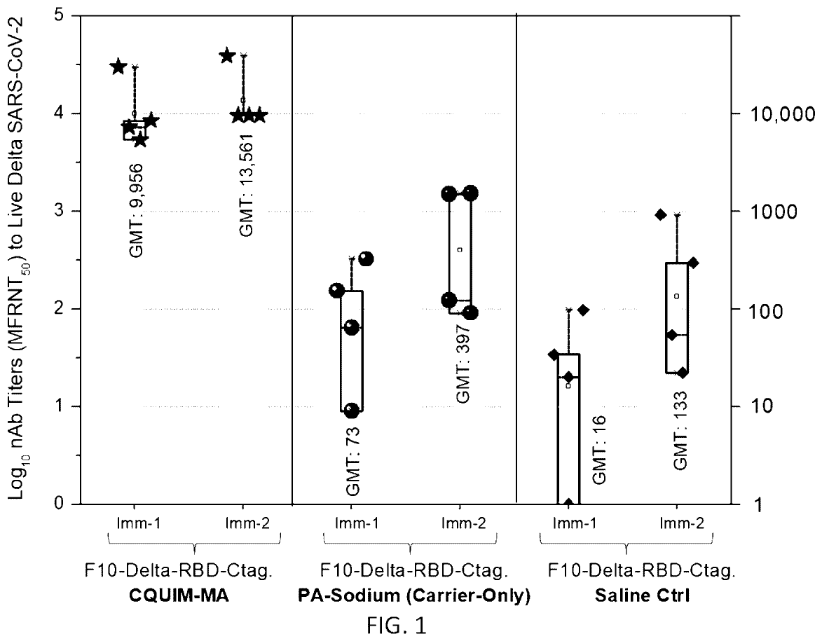

Rabbits were immunized with a ferritin nanoparticle-based SARS-COV-2 RBD (Delta) antigen, adjuvanted with either the adjuvant compound (n=4), or poly (acrylic acid) as carrier control (n=4), or saline alone control (n=4). An antigen concentration of 20 micrograms/dose, and an adjuvant concentration of 20 micrograms/dose of imidazoquinoline content (e.g., for the polymeric adjuvant) was used in a total volume of 0.2 mL/nostril. The vaccine was aerosolized into each nostril using a Mucosal Atomization Device. Rabbits were primed on Day 0, and boosted on Days 15 and 28. Pre-immune and post-boost-1 (“Immune-1”) and post-boost-2 (“Immune-2”) sera were collected and analyzed for neutralizing antibodies against live SARS-COV-2 virus (Delta variant) using rapid-throughput, high-content, Microfocus Reduction Neutralization Tests (MFRNT) developed by ViroVax. As shown in FIG. 1, the intranasal vaccine construct adjuvanted with the polymeric adjuvant elicits very high systemic neutralizing antibodies against live Delta SARS-COV-2 after a single boost, resulting in a geometric mean titer of 9,956.

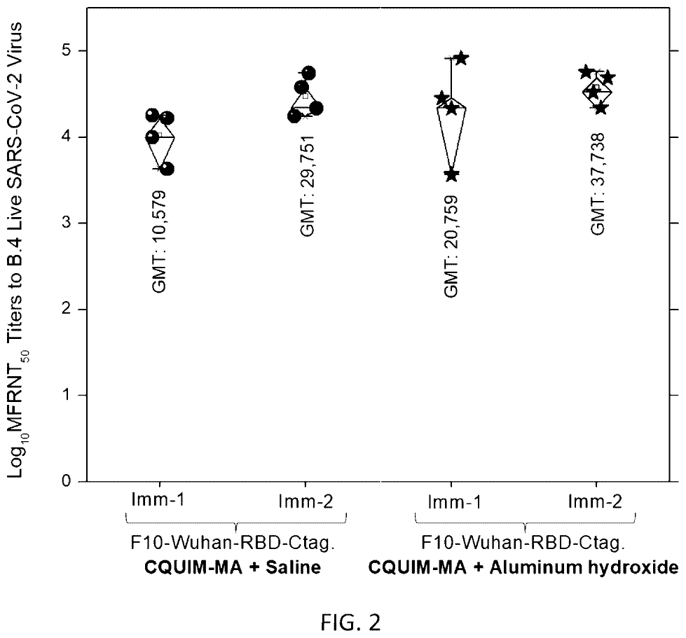

Adjuvant for Intramuscular Subunit Vaccines (Rabbit Immunogenicity Trial VV-40) Rabbits were immunized with a ferritin nanoparticle-based SARS-COV-2 RBD (Wuhan) antigen, adjuvanted with either the polymeric adjuvant in the absence of aluminum hydroxide (n=4), or the polymeric adjuvant in the presence of 200 micrograms of aluminum hydroxide. An antigen concentration of 20 micrograms/dose, and an adjuvant concentration of 20 micrograms/dose of imidazoquinoline content (e.g., for the polymeric adjuvant) was used in a total volume of 0.2 mL. The vaccine was administered intramuscularly. Rabbits were primed on Day 0, and boosted on Days 15 and 28. Pre-immune and post-boost-1 (“Immune-1”) and post-boost-2 (“Immune-2”) sera were collected and analyzed for neutralizing antibodies against live SARS-COV-2 virus (B.4 “Iranian” variant) using the MFRNT. As shown in FIG. 2, the polymeric adjuvant shows excellent adjuvant activity, and elicits very high neutralizing antibodies even in the absence of aluminum hydroxide. These results establish that (1) the polymeric adjuvant can be used as a standalone adjuvant in alum-free vaccine formulations as lyophilizates, thereby greatly extending the shelf-life of the vaccine; and (2) the polymeric adjuvant can be used as an add-on adjuvant for pre-existing “alum”-adsorbed vaccines.
Superiority of the adjuvant compound over Alhydroxiquim-II for Intramuscular Subunit Vaccines (Rabbit Immunogenicity Trial VV-45)

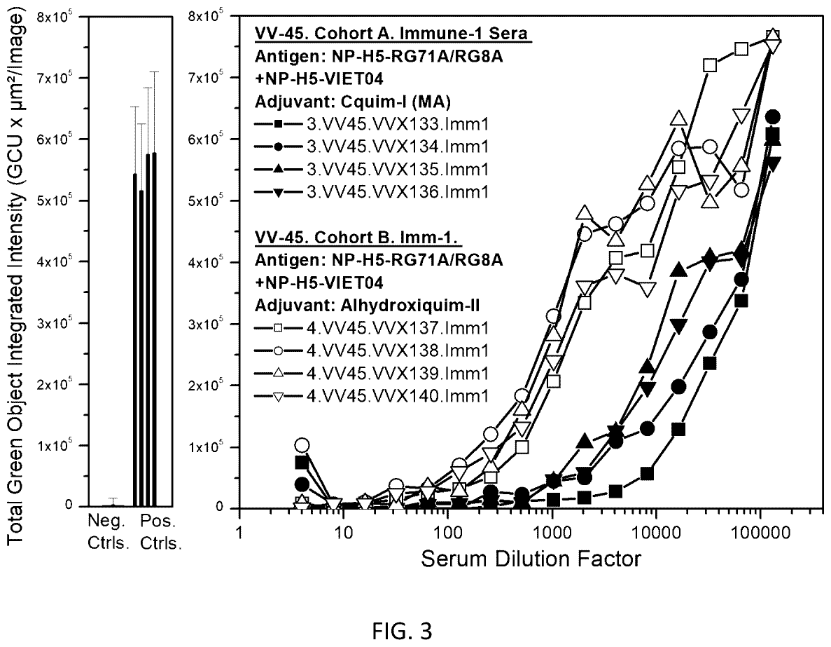

Rabbits were immunized with a ferritin nanoparticle-based Influenza H5 antigens corresponding to the ectodomains of Clade 1 and Clade 2.3.3.4b hemagglutinin adjuvanted with either the polymeric adjuvant or the Alhydroxiquim-II, each containing 20 micrograms imidazoquinoline/dose. An antigen concentration of 20 micrograms/dose was used in a total volume of 0.2 mL. The vaccine was administered intramuscularly. Rabbits were primed on Day 0, and boosted on Days 15 and 28. Pre-immune and post-boost-1 (“Immune-1”) and post-boost-2 (“Immune-2”) sera were collected and analyzed for neutralizing antibodies against Green fluorescent protein-expressing, replication-incompetent, pseudotyped viral particles using the MFRNT. As shown in FIG. 3, the neutralizing antibody titers elicited by the polymeric adjuvant (GMT: 11,018) are approximately an order of magnitude greater than that elicited by Alhydroxiquim-II (Geometric Mean Titer: 990), demonstrating the superiority of the polymeric adjuvant as an adjuvant over Alhydroxiquim-II.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

1. A compound comprising the structure of Formula 1: 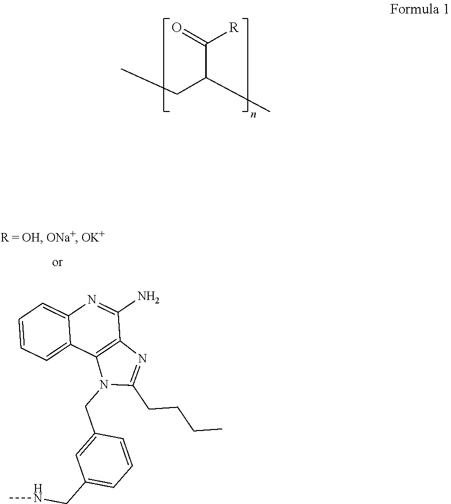
wherein at least one 1-(3-(aminomethyl)benzyl)-2-butyl-1H-imidazo[4,5-c]quinolin-4-amine is conjugated via the meta-aminomethylbenzyl substituent at position N1 of the imidazoquinoline to poly (acrylic acid) via an amide bond, and ‘n’ is an integer.

2. The compound of claim 1, wherein a degree of conjugation of the imidazoquinoline moiety to poly (acrylic acid) is from about 5% to about 50%.

3. A composition comprising:
the compound of claim 1;
an immunogen; and
a pharmaceutically acceptable carrier.

4. The composition of claim 3, containing, in addition, alum particles.

5. The composition of claim 3 in a lyophilized form.

6. A method of adjuvating an immunological response, comprising:
administering the composition of claim 3 or claim 4 to a subject either parenterally, or intranasally.

7. A method of preparing the compound of claim 1, comprising:
conjugating at least one 1-(3-(aminomethyl)benzyl)-2-butyl-1H-imidazo[4,5-c]quinolin-4-amine to the poly (acrylic acid).
